(19) United States
(12) Patent Application Publication
(10) Pub. No.: US 2013/0143933 A1
(43) Pub. Date: Jun. 6, 2013

(54) TARGETED CORRECTION OF A GENETIC DEFECT IN CANCER THERAPY

(71) Applicant: TRT Pharma Inc., Montreal (CA)

(72) Inventors: Gérald BATIST, Montreal (CA); Jian Hui WU, Montreal (CA)

(73) Assignee: TRT Pharma Inc., Montreal (CA)

(21) Appl. No.: 13/673,090

(22) Filed: Nov. 9, 2012

Related U.S. Application Data
(60) Provisional application No. 61/557,646, filed on Nov. 9, 2011.

(51) Int. Cl. C07D 213/81; A61K 31/4409; C07C 323/48; C07C 251/86; C07C 243/00; A61K 31/175; C07D 213/86

(52) U.S. Cl. CPC C07D 213/81; C07C 323/48; C07C 251/86
USPC 514/354; 564/150; 564/149; 546/325; 514/615

(57) ABSTRACT

The present document describes a cancer mutation-selective chemosensitizer that comprise compounds for restoring association between mutated keap1 protein and Nrf2 protein, and inhibition of Nrf2 functions. The present document also describes composition of matter containing the compounds, as well as methods of medical treatment for treating diseases such as cancer with the compounds.

### CROSS-REFERENCE TO RELATED APPLICATIONS

This application claims priority of U.S. provisional patent application U.S. 61/557,646, filed 9 Nov. 2011, the specification of which is hereby incorporated by reference.

### BACKGROUND

(a) Field

The subject matter disclosed generally relates to a mutation-selective chemosensitizer for overcoming therapeutic resistance to chemotherapy, and more specifically to mutation selective chemosensitizers for correcting a series of Keap1 mutations to restore the interaction between a mutated Keap1 protein and a Nrf2 protein, for inhibiting the activity of Nrf2.

(b) Related Prior Art

Therapeutic resistance remains a cause of cancer deaths. It is clear that to have clinical impact a successful strategy must target more than a single mechanism of resistance, and also provide tumor selectivity to avoid enhancing normal tissue toxicity. The transcriptional protein Nrf2 regulates multiple mechanisms of cytoprotection such as ABCC1 (efflux pump), detoxification and drug metabolism enzymes, glutathione synthesis and other mechanisms to protect from oxidant stress.

NF-E2 p45-related factor 2 (Nrf2) is increasingly recognized as central to resistance to cytotoxic drugs, radiation therapy and also to some targeted agents like EGFR small molecule inhibitors. Depletion of cellular Nrf2 levels in cell lines, using shRNA or small molecules confirms the role of Nrf2 in therapeutic resistance. Low Nrf2 in breast cancer cell lines and in the majority of a 200-sample tissue microarray is consistent with the high response rates of breast cancer to many cytotoxic therapies. Cell lines engineered to selectively increase Nrf2 levels have enhanced detox mechanisms and dramatic cellular resistance to relevant drugs. Translating these findings into therapeutic interventions is difficult since both siRNA and small molecule inhibitors could enhance systemic toxicity if they deplete Nrf2 in both tumor and normal cells.

Under basal conditions, the redox-sensitive protein Kelch-like ECH-associated protein 1 (Keap1) binds Nrf2 to form a Keap1/Nrf2 complex, and anchors it in the cytoplasm. Keap1 is an adaptor protein for the Cullin 3 ubiquitin E3 ligase (CuI3), and specifically targets Nrf2 for degradation by the ubiquitin-proteasome pathway. With oxidative stress, Keap1 undergoes conformational changes that disrupt its interaction with Nrf2, which can then translocate to the nucleus and activate the cytoprotective program. In both cell lines and clinical specimens of non-small cell lung cancer (NSCLC), loss-of-function Keap1 mutations result in constitutively high levels and active Nrf2 and subsequent resistance to chemotherapeutic drugs (taxanes, platinums) and radiotherapy. Keap1 mutations are reported in up to 60% of papillary lung adenocarcinoma, as well as in other tumors including ovarian, gall bladder and others.

Another mechanism of post-translational regulation of Nrf2 has been described, involving the serine/threonine glycogen synthetase kinase 3 (GSK-3β), a protein that regulates glycolytic metabolism and is a downstream target in the Phosphoinositide-3 Kinase (PI3K) signaling pathway. In this scenario, GSK-3β acts as an adapter protein for Nrf2, targeting it to the SCF/{beta}-TrCP SCF protein complex for ultimate ubiquination and proteosomal degradation. This is a Keap1 independent process. Activation of the PI3Kinase signaling pathway, whether by mutation or increased copy number of PI3K and other genes in the pathway (PTEN, Akt), is found relatively frequently in a variety of cancers. Phosphorylation of GSK-3β actually inactivates its enzymatic function, while inhibitors of this pathway, many of which are in clinical development, would have the relatively selective effect of increasing GSK-3β activity in these tumors, providing an opportunity for this alternative Nrf2 degradation pathway to play a role.

There is a need for selectively depleting Nrf2 in tumor cells and producing highly targeted chemosensitization.

Therefore, there is a need for molecules that would inhibit Nrf2 activity and prevent, or at least negatively influence its activity.

### SUMMARY

According to an embodiment, there is provided a mutation-selective chemosensitizer comprising a compound of formula (I) for opening a mutated Nrf2 binding site of a mutated keap1 protein to restore interaction between the mutated keap1 protein and a Nrf2 protein:

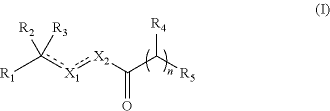

wherein R₁ may be a heterocyclic aromatic or non-aromatic 5 to 8-membered ring or double-ring containing 1-8 heteroatoms selected from N, O or S,
wherein N and S may be oxidized and N may be quaternized, and any atom of said ring can be substituted with a group chosen from
i. halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
ii. heterocyclic aromatic or non-aromatic 5- to 10-membered ring containing 1-4 heteroatoms selected from N, O or S, wherein N and S may be oxidized and N may be quaternized,
iii. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂,

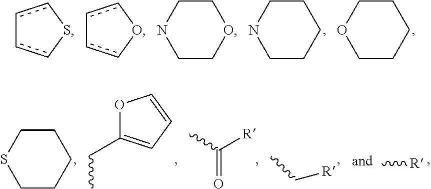

wherein R′ may be chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
   R₅ may be selected from
iv. hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
v. heterocyclic aromatic or non-aromatic 5- to 10-membered ring containing 1-4 heteroatoms selected from N, O or S, wherein N and S may be oxidized and N may be quaternized,
vi. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂,

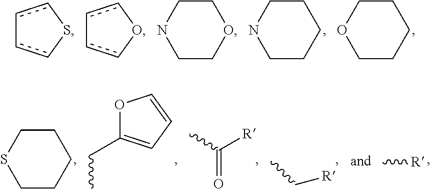

wherein R′ may be chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
Vii.

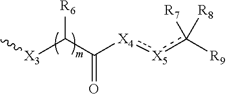

R₂, R₃, R₄, R₆, R₇, and R₈ may be independently chosen or identical, and may be chosen from
viii. hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
ix. heterocyclic aromatic or non-aromatic 5- to 10-membered ring containing 1-4 heteroatoms selected from N, O or S, wherein N and S may be oxidized and N may be quaternized,
x. ═O, CH₃, OCH₃, OC₂H₅, NO₂, ON, F, Cl, Br, SH, CF₃, OCF₃, O(CF₂)₂H, NH₂, N(CH₃)₂, N(C₂H₄OH)₂, CH(OC₂H₅)₂,

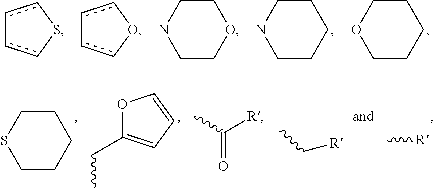

wherein R′ may be chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
   R₉ may be a heterocyclic aromatic or non-aromatic 5 to 8-membered ring or double-ring containing 1-8 heteroatoms selected from N, O or S, wherein N and S may be oxidized and N may be quaternized, and any atom of said ring may be substituted with a group chosen from
xi. halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
xii. heterocyclic aromatic or non-aromatic 5- to 10-membered ring containing 1-4 heteroatoms selected from N, O or S, wherein N and S may be oxidized and N may be quaternized,
xiii. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂,

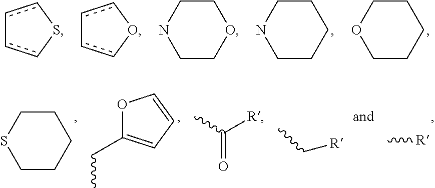

wherein R′ is chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
   X₁, X₂, X₄ and X₅ may be independently chosen or identical and may be selected from N, NH, C, CH or CH₂,
   X₃ may be selected from N, O, or S,
   n and m may be independently chosen or identical and may be 1 to 10 C atom,
   wherein [symbol] indicates an attachment point, and [symbol]
   may be a single bond or a double bond;
   pharmaceutically acceptable salt, racemic mixture, enantiomer, diastereoisomer, isomer, and tautomer thereof.

The compound may be a compound of formula (II):

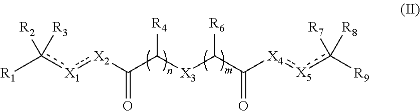

wherein R₁, R₂, R₃, R₄, R₅, R₆, R₇, R₈, R₉, X₁, X₂, X₃, X₄, and X₅ are as defined above.

The compound may correct a Keap1 mutation to restore interaction between a mutated Keap1 protein and the Nrf2 protein.

The compound of formula (II) may be

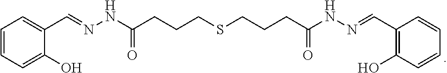 [SMILES: O=C(CCCSCCCC(=O)N/N=C/c1ccccc1O)N/N=C/c1ccccc1O]

The compound of formula (II) may be

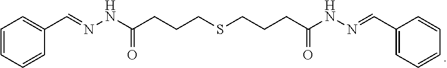 [SMILES: O=C(CCCSCCCC(=O)N/N=C/c1ccccc1)N/N=C/c1ccccc1]

The compound of formula (II) may be

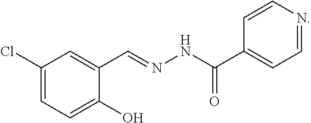 [SMILES: O=C(N/N=C/c1cc(Cl)ccc1O)c1ccncc1]

The compound of formula (II) may be

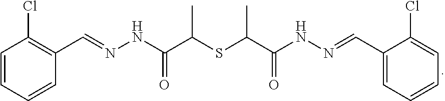 [SMILES: CC(SC(C)C(=O)N/N=C/c1ccccc1Cl)C(=O)N/N=C/c1ccccc1Cl]

The compound of formula (II) may be

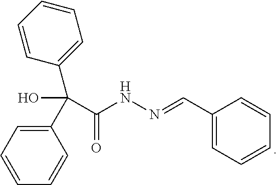 [SMILES: O=C(N/N=C/c1ccccc1)C(O)(c1ccccc1)c1ccccc1]

The compound of formula (II) may be

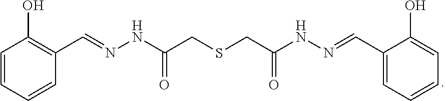 [SMILES: O=C(CSCC(=O)N/N=C/c1ccccc1O)N/N=C/c1ccccc1O]

According to another embodiment, there is provided a pharmaceutical composition for the inhibition of a Nrf2 protein which comprises a therapeutically effective amount of a compound of formula (I), or (II) as defined above, in association with a pharmaceutically acceptable carrier.

According to another embodiment, there is provided a pharmaceutical composition for overcoming drug resistance in cancer chemotherapy and for the inhibition of tumor growth which comprises a therapeutically effective amount of a compound of formula (I), or (II) as defined above, in association with a pharmaceutically acceptable carrier.

According to another embodiment, there is provided a method of treating and/or preventing a disease which involves the abnormal activation or expression of a Nrf2 protein comprising administering a therapeutically effective amount of the compound of formula (I), or (II) as defined above.

According to another embodiment, there is provided a method of treating a cancer in a subject in need thereof comprising administering a therapeutically effective amount of a compound of formula (I), or (II) as defined above.

The cancer may be chosen from liver cancer, lung cancer, breast cancer, prostate cancer, colon cancer, neuroblastoma or leukemia.

The following terms are defined below.

The term “chemosensitize” is intended to mean to make sensitive or reactive to a chemical agent (e.g. a chemotherapeutic agent for treating cancer) to which the tumor or diseased cells or organ responded poorly to.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates that molecular dynamic simulations showed that the peptide derived from Nrf2 is drifting away from the Keap1 in the G350S mutant (green ribbon) and R413L mutant (blue ribbon), but not the wild type (red ribbon). The WT/peptide, G350S/peptide and R413L/peptide complexes shared the same initial structure and are subjected to molecular dynamic simulations with exact the same protocol for 9 ns. Relative to the wild type Keap1, the backbone root mean square deviations (RMSD) for the G350S and R413L mutants are 1.05 and 1.17 Å, respectively. Our simulations revealed that G350S and R413L mutations in Keap1 are disrupting the association between Keap1 and Nrf2, consistent with experimental findings.

FIG. 2 illustrates a schematic representation of the strategy for restoring association of multiple mutated Keap1 with Nrf2.

FIG. 3 illustrates that compound NR16 suppresses A2ARE-dependent reporter activity in A549 cells in a dose-dependent manner and that NR13 is inactive in the reporter assay.

FIG. 4 illustrates that there is a dose-dependent decrease (NR16 at 1 and 5 μM) in Nrf2, compared to DMSO and that NR13 is inactive in the western blot analysis.

FIG. 5 illustrates the core structure of NR16, identified using the in silico and luciferase inhibition screening steps.

FIG. 6 illustrates that (A) in H460 cells which bear both mutations in PI3K (E545K) and Keap1 (D286H), exposure of the cells to the dual and potent PI3K and mTOR inhibitor PF-04691502 cause within 6 hours a dramatic drop in phospho-GSK at the critical Ser9 position. Within 24 hours cellular Nrf2 is reduced. (B) This was not observed even at a higher dose in A549 cells, which have wild type PI3K.

FIG. 7 illustrates that (A) PF-04691502 sensitizes H460 cells to paclitaxel in vitro, using two concentrations of PF (10 and 50 nm) and a range of paclitaxel doses. Cell survival was measured using AlamarBlue and fluorescence detection for viable (metabolically active) cells with 560Exnm/590EMnm filters. The plotted fluorescence intensity versus paclitaxel concentration as a percentage of control (no paclitaxel) shows the significant sensitization to paclitaxel with exposure to PF-04691502. (B) The same experiment in NSCLC cell line A549, which has a Keap1 mutation but wild type PI3K, shows no sensitization. Same was the case in MCF-7 cells, which bear a PI3K mutation but no Keap1 mutation.

FIG. 8 illustrates a titration curve obtained by co-transfecting cells with a fixed amount of Nrf2-Rluc (500 ng), and a range of Keap1-GFP2 amounts (10, 50, 100, 500 and 1000 ng).

FIG. 9 illustrates clones transfected with siRNAs directed to GSK-3R or Keap1.

FIG. 10 illustrates that NK20, NK22, NK23 and NK24 at 5 μM suppressed A2ARE-luc reporter activity in A549 cells.

### DETAILED DESCRIPTION OF THE PREFERRED EMBODIMENTS

In embodiments there are disclosed compounds that are highly selective for the depletion of Nrf2 in tumor. Somatic mutations of genes involved in Nrf2 post-translational regulation are believed to be highly selective targets to sensitize tumors to anti-cancer therapies.

### Correcting Keap1 Mutations: Mutation-Selective Chemosensitizers

According to an embodiment, there are disclosed mutation-selective chemosensitizers for correcting Keap1 mutations. The design of chemical compounds that restore the association of mutant Keap1 with Nrf2 is believed to be a remarkably selective way to sensitize tumor cells. Molecular dynamic simulations, virtual screening and targeted biological and biophysical assays are used to achieve this goal.

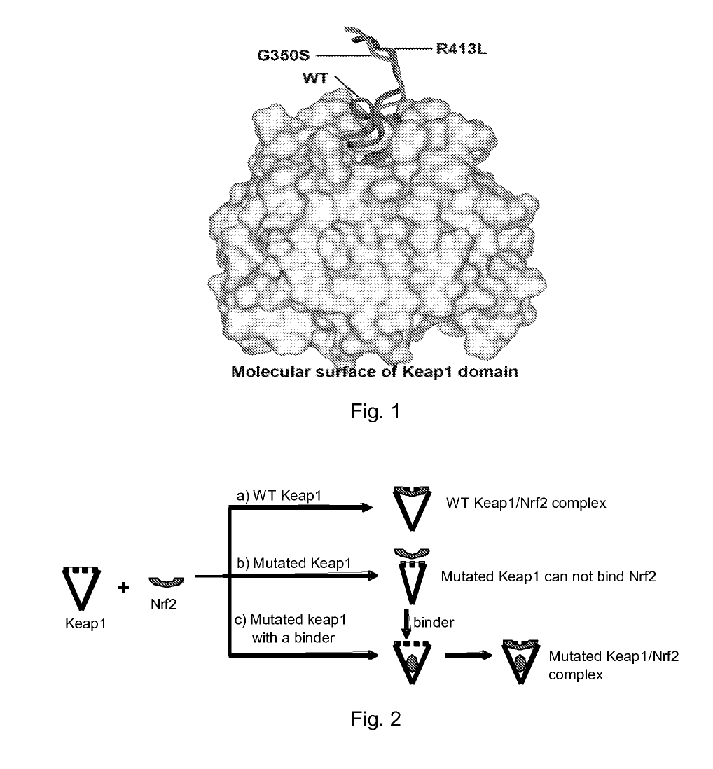

The crystal structure of Keap1/Nrf2 interface (PDB entry: 2flu) provides a solid basis for computational simulations to detect the structural impact of point mutations of Keap1 on the Keap1/Nrf2 complex, and to search for a novel strategy for restoring the association of the mutated Keap1 with Nrf2. To detect the mutation-induced structural changes at the interface of Keap1/Nrf2, molecular dynamics (MD) simulations are performed for the following three complexes: i) wild type Keap1/Nrf2 complex (WT/Nrf2), ii) G350S mutated Keap1/Nrf2 complex (G350S/Nrf2), and iii) R413L mutated Keap1/Nrf2 complex (R413L/Nrf2), using software AMBER (UCSF, CA, USA). The above three Keap1/Nrf2 complexes share the same initial structure, which are generated from the crystal structure of keap1/Nrf2 complex (PDB entry: 2flu). The MD simulations indicate that the Nrf2 peptides at G350S/Nrf2 and R413L/Nrf2 drift away from the mutated Keap1, consistent with previous experimental findings that G350S and R413L mutated Keap1 are incapable of binding with Nrf2. In contrast, Nrf2 peptide at the WT/Nrf2 remains stable, suggesting the Nrf2 drift away is due to the Keap1 mutations (FIG. 1). Importantly, both G350S and R413L mutations have similar effect on the Keap1 binding site at the Keap1/Nrf2 interface.

The Keap1 binding site can be viewed as a ‘V’ shaped cavity (FIG. 2). The simulations indicate that Keap1 mutations lead to a narrow opening of the ‘V’ shape (i.e., short dashed line), which in turn prevents the formation of a complex between mutated Keap1 and Nrf2. While G350S and R415G are far away from each other in the 3D structure, they are predicted to generate a similar effect on the Keap1 binding site. These results inspired a novel strategy to design chemical compounds to restore association of mutated Keap1 and Nrf2, as follows.

Since wild type Keap1 with a correct opening of the ‘V’ shape (dash line) is capable of forming the Keap1/Nrf2 complex; it is believed that a chemical binder that binds the lower portion of the ‘V’ shape could hold the ‘V’ shape opening of the mutated Keap1 at the correct size for Nrf2 binding, and thus restores the association of the mutated Keap1 with Nrf2. It is believed that it is feasible for a chemical compound to be effective at restoring association of multiple mutated Keap1 with Nrf2 so long as the compound holds the Keap1 ‘V’ shape opening at the correct size, as schematically represented in FIG. 2.

To test this strategy, the NCI OPEN Chemical database is virtually screened for chemical compounds that bind the CTD of Keap1 to restore the association of Keap1 and Nrf2, using software GOLD (CCDC, Cambridge, UK). 18 top-score chemical candidates are obtained from NCI, US chemical libraries, and screened these with an ARE-dependent reporter assay in A549 cells, as shown below.

### ARE-Dependent Luciferase Assays in A549 Cells

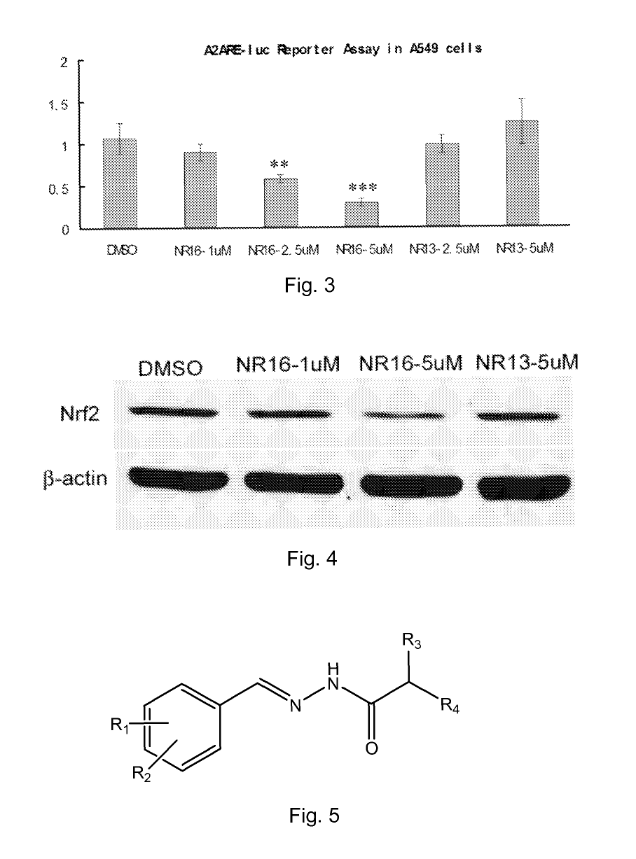

A549 cells are highly resistant to carboplatin and constitutively express high level of Nrf2 due to a mutation (G333C) in Keap1. Using ARE-dependent luciferase assays in A549 cells, which have constitutively high Nrf2 levels, the 18 compounds that are designed to target the CTD of Keap1 are tested. Included amongst these are NR16 and NR13. FIG. 3 demonstrates that compound NR16 suppressed A2ARE-dependent reporter activity in A549 cells in a dose-dependent manner. A2ARE-luc plasmid is transiently transfected into A549 cells. pRL-TK is included as internal control. Transfected cells are treated with DMSO vehicle, compound NR16 at 1, 2.5 and 5 μM as well as NR13 at 2.5 μM for 24 hours. **p<0.005, ***p<0.0005 when compared with DMSO vehicle.

This effect is mediated by a depletion of Nrf2, as confirmed by immunoblotting of Nrf2 protein levels in A549 cells, exposed to NR16 for 48 hours (FIG. 4). There is a dose-dependent decrease (NR16 at 1 and 5 μM), compared to DMSO. NR13 is inactive in the reporter assay and western blot analysis.

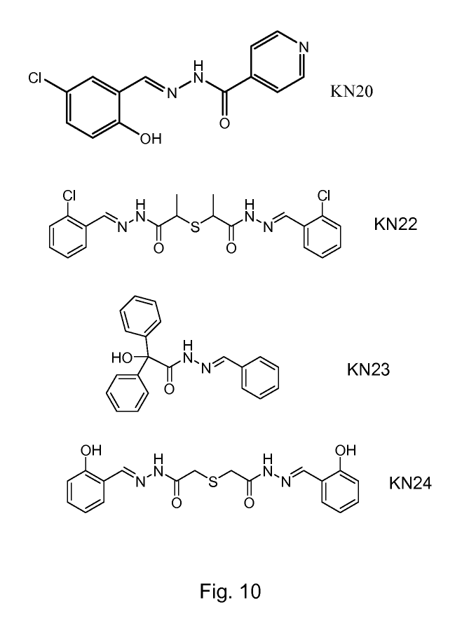

Similarly, FIGS. 10A-B demonstrates that compounds KN-20, KN-22, KN-23, and KN-24 suppressed A2ARE-dependent reporter activity in A549 cells. A2ARE-luc and pRL-TK plasmids are transiently transfected into A549 cells. 6 hours after transfection, the transfected cells are exposed to DMSO vehicle control or compound at designated doses (5 μM) for 24 hours. ***p<0.0001 when compared with vehicle control.

### Core Structure of Chemosensitizer Compound

FIG. 5 shows the core structure of NR16, identified using the in silico and luciferase inhibition screening steps described. There are multiple sites at which different substitution groups can be added to optimize the potency of NR16:

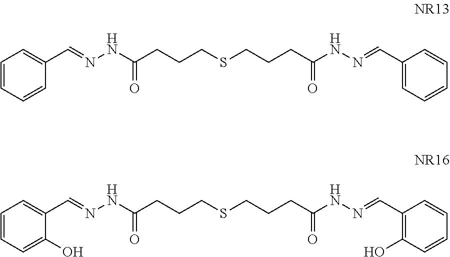 [SMILES: O=C(CCCSCCCC(=O)N/N=C/c1ccccc1)N/N=C/c1ccccc1 ; O=C(CCCSCCCC(=O)N/N=C/c1ccccc1O)N/N=C/c1ccccc1O]

NR13 is a close analogue of NR16 differing in only one OH group. This suggests it is possible to optimize the potency of NR16 by attaching various

R groups to the core structure of NR16. FIG. 10B shows the structure of compounds KN-20, KN-22, KN-23, and KN-24.

According to an embodiment, there are disclosed chemosensitizer compounds. The mutation-selective chemosensitizer compounds may be compound of formula (I) for opening a mutated Nrf2 binding site of a mutated keap1 protein to restore interaction between said mutated keap1 protein and a Nrf2 protein:

According to another embodiment, the mutation-selective chemosensitizer compound may be correcting a Keap1 mutation to restore interaction between the mutated Keap1 protein and said Nrf2 protein.

According to another embodiment of the present invention, there is disclosed mutation-selective chemosensitizers compounds of formula (I) for opening a mutated Nrf2 binding site of a mutated keap1 protein to restore interaction between the mutated keap1 protein and a Nrf2 protein:

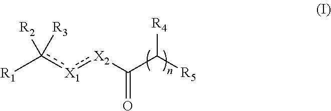

According to some embodiment, the R₁ may be a heterocyclic aromatic or non-aromatic 5 to 8-membered ring or double-ring containing 1-8 heteroatoms selected from N, O or S, and the N and S can be oxidized and N can be quaternized, and any atom of the ring can be substituted with a group chosen from the following:
i. halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
ii. heterocyclic aromatic or non-aromatic 5- to 10-membered ring containing 1-4 heteroatoms selected from N, O or S, wherein N and S can be oxidized and N can be quaternized,
iii. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂,

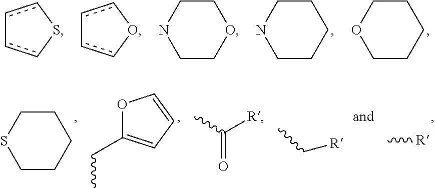

wherein R′ may be chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
   R₅ may be selected from the following:
iv. hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
v. heterocyclic aromatic or non-aromatic 5- to 10-membered ring containing 1-4 heteroatoms selected from N, O or S, wherein N and S can be oxidized and N can be quaternized,
vi. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂,

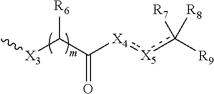

wherein R′ may be chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
Vii.

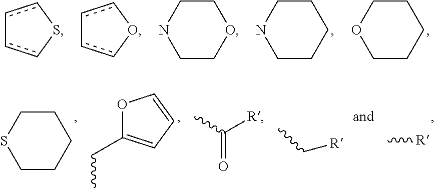

R₂, R₃, R₄, R₆, R₇, and R₈ are independently chosen or identical, and are chosen from
viii. hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
ix. heterocyclic aromatic or non-aromatic 5- to 10-membered ring containing 1-4 heteroatoms selected from N, O or S, wherein N and S can be oxidized and N can be quaternized,
x. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂,

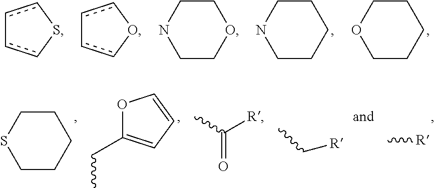

wherein R′ may be chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
   R₉ may be a heterocyclic aromatic or non-aromatic 5 to 8-membered ring or double-ring containing 1-8 heteroatoms selected from N, O or S, and N and S can be oxidized and N can be quaternized, and any atom of the ring can be substituted with a group chosen from the following:
xi. halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
xii. heterocyclic aromatic or non-aromatic 5- to 10-membered ring containing 1-4 heteroatoms selected from N, O or S, wherein N and S can be oxidized and N can be quaternized,
xiii. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂,

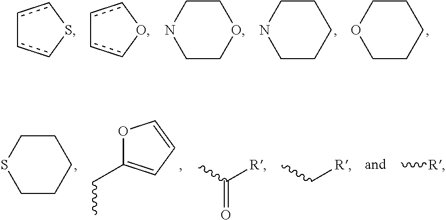

wherein R′ may be chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
   X₁, X₂, X₄ and X₅ may be independently chosen or identical and may be selected from N, NH, C, CH or CH₂,
   X₃ may be selected from N, O, or S,
   n and m may be independently chosen or identical and can be 1 to 10 C atom, [symbol]
   indicates an attachment point, and [symbol] is a single bond or a double bond; pharmaceutically acceptable salt, racemic mixture, enantiomer, diastereoisomer, isomer, and tautomer thereof.

According to another embodiment, the mutation-selective chemosensitizer may be a compound of formula (II):

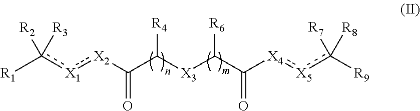

and R₁, R₂, R₃, R₄, R₅, R₆, R₇, R₈, R₉, X₁, X₂, X₃, X₄, and X₅ are as defined above.

### Enhancing GSK-31β-Mediated Nrf2 Depletion in the Presence of Keap1 Mutations.

According to another embodiment, the proposed enhancement is selective of tumors vs. normal cells, as it is relying on the presence of two somatic genetic events. Enhanced PI3Kinase signaling, may be due to increased gene copy number or mutations, and is relatively common in a variety of cancers. Activation of this pathways leads to phosphorylation of GSK-3β at its Ser9 position, which deactivates GSK-3β function. These tumors have low basal GSK-3β activity. Thus, in the presence of Keap1 mutations PI3Kinase inhibitors may enhance GSK-3β-mediated Nrf2 degradation.

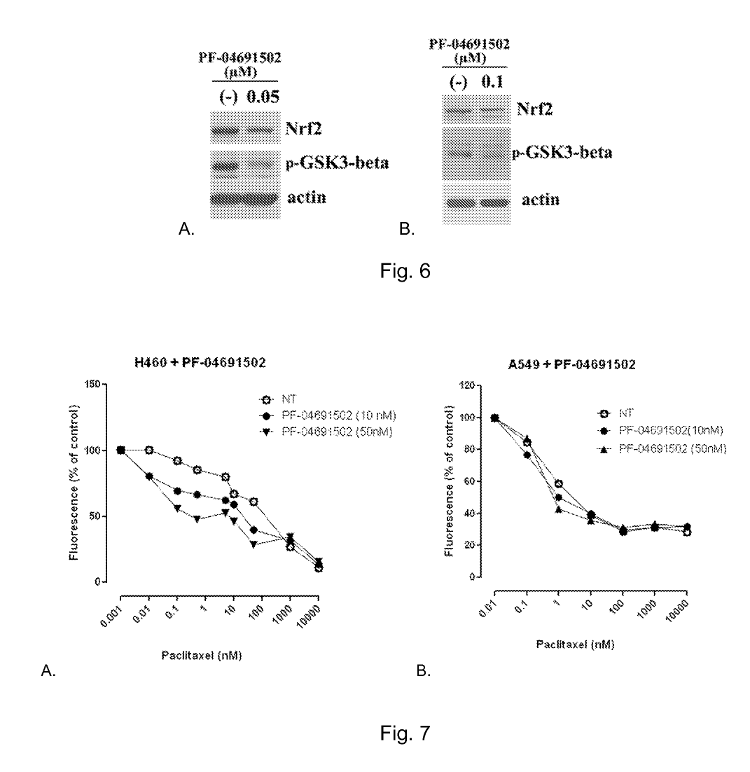

H460 cells, which bear both mutations in PI3K (E545K) and Keap1 (D286H) are exposed the cells to the dual and potent PI3K and mTOR inhibitor PF-04691502 currently in clinical development. Within 6 hours a dramatic drop in phospho-GSK at the critical Ser9 position is detected (FIG. 6A). Within 24 hours cellular Nrf2 is reduced (FIG. 6A). This is not observed even at a higher dose in A549 cells, which have wild type PI3K (FIG. 6B).

PF-04691502 is tested to determine is it sensitizes H460 cells to paclitaxel in vitro, using two concentrations of PF (10 and 50 nm) and a range of paclitaxel doses. Cell survival is measured using AlamarBlue and fluorescence detection for viable (metabolically active) cells with 560Exnm/590EMnm filters. The plotted fluorescence intensity versus paclitaxel concentration as a percentage of control (no paclitaxel) shows that significant sensitization to paclitaxel with exposure to PF-04691502 is obtained (FIG. 7A). The same experiment in NSCLC cell line A549, which has a Keap1 mutation but wild type PI3K, shows no sensitization (FIG. 7B). MCF-7 cells which bear a PI3K mutation but no Keap1 mutation show no sensitization.

Identification of the Patient/Tumor Population with the Somatic Mutations for Chemo-Sensitization.

The presence of Keap1 somatic mutations, alone and with genetic alterations in PI3Kinase expression represent potential ‘molecular signatures’ of tumors that may be selectively sensitized to cytotoxic chemotherapy using the strategies proposed herein. These might be found in a relatively small subset of patients with different tumor types. This is in fact an emerging paradigm that has evolved in the course of the development of some highly effective therapies (in biomarker positive patients') such as trastuzumab (Herceptin). In that case, only ˜15% of breast cancers have amplified HER-2 receptor, but in a population composed exclusively of patients with such tumors, the clinical benefit is enormous. This appears to be the case for a smaller number of gastric cancers which also have HER2 amplification. Another example is Crizotinib, a novel small molecule that is effective in NSCLC bearing the ALK-EML4 fusion gene, which represents no more than 5% of NSCLC, and an even smaller number of sarcomas. Therefore, the prediction that Nrf2-directed strategies will be active and highly selective in a relatively small sub-set of a variety of tumors is entirely consistent with the current evolution of molecularly-based therapeutics in cancer.

These observations are made in clinical samples associating Nrf2 levels due to loss-of-function somatic mutations in Keap1 with therapeutic resistance, all confirmed in in vitro models. These studies are extended to explore potential strategies for highly selective sensitization by Nrf2 depletion. Initial screens of the Keap1 sequences in cell lines and some clinical specimens, crossing diagnostic boundaries and using methodology that is easily transferred to a clinical diagnostic setting, i.e. accurate and reproducible in testing formalin-fixed and paraffin embedded (FFPE) samples, which often yields degraded DNA. High Resolution Melting (HRM) curve analysis is used to scan for mutations as an initial screen, and potential positives are validated using direct sequencing. Since the optimal size of the PCR product for sensitive HRM analysis is between 150 to ˜300 bp, and the Keap1 exon of interest is 686 bp, it is digested into 3 overlapping fragments and each analyzed. Keap1 mutations are confirmed in NSCLC lines H460 (D286H) and A549 (G333C), and PI3K mutation at (E545K) in H460 and not in A549. Both assays are validated in a series of clinical specimens, starting with NSCLC, registered in the COSMIC Sanger database as the most frequent tumor with Keap1 mutations. It is quite uncommon in breast cancer (3 mutations in 222 samples tested), although a very recent study of 30 cases of ovarian cancer found mutations in 29% of clear cell samples and 8% of non-clear cell tumors, and 30% in a series of gall bladder tumors. Only small numbers of other tumors have been tested to date. Keap 1 mutation at Y255H have been identified in the hormone-resistant human prostate cancer cell line LnCAP, which has constitutively activated Akt, which results in downstream phospho-GSK-3β.

### Characterization and Optimization of Compound NR16 as a Novel Selective Nrf2 Inhibitor

The A549 cells have constitutively elevated Nrf2 due to Keap1 mutations. To confirm that the effect of NR16 and derivatives is mediated by Nrf2-ARE interaction the ARE-luc assay is repeated using a mutant ARE-luc. Next, A549 cells are exposed to NR16 for 48 hours as well as to the proteosome inhibitor MG132 10 μM, and cell lysates are immunoprecipitated with an Nrf2 antibody, and the result detected with antibodies that detect ubiquitin (Epitomics, AbCam, R&D Systems).

To confirm that NR16 and derivatives restore the association of the G333C mutated Keap1 and Nrf2, H838 cells which express a truncated Keap1 and elevated level of Nrf2 are used. They are transiently transfected with A2ARE-luc reporter alone and in combination with mutant (G333C) Keap1-expressing plasmids into. The transfected cells are treated with control DMSO and compound NR16.

While Keap-Nrf2 interactions have been demonstrated indirectly by various means, direct interaction using the bioluminescence resonance energy transfer (BRET2) assay has been established, which provides real-time monitoring of protein-protein interactions in living cells (30). This assay is used to determine the effect of NR16 and derivatives. One protein is fused to the energy donor renilla luciferase (Rluc) and the second protein is fused to the energy acceptor green fluorescent protein (GFP2). BRET2 is used to assess Nrf2/Keap1 interaction. Nrf2 is genetically fused to Rluc, while Keap1 is fused to GFP2. As a negative control, a keap1 mutant that does not bind Nrf2 is used. RLUC-Nrf2 and GFP2-Keap1 encoding plasmids are transfected in H460 cells, and 48 hours after later, cells are collected. About 200.000 cells from each transfection are plated in 96 wells plate. The coelenterazine analog DeepBlueC (5 μM) is used as a substrate for Rluc, which emits blue light peaking at 400 nm. Then, excitation of GFP2 by RLUC results in emission of green light at 510 nm. Energy transfer efficiencies between Rluc/DBC and GFP2 are determined ratiometrically as a 510/400 ratio. This measurement is referred to as the BRET2 signal and reflects the proximity of RLUC to GFP2, and of Keap1 to Nrf2.

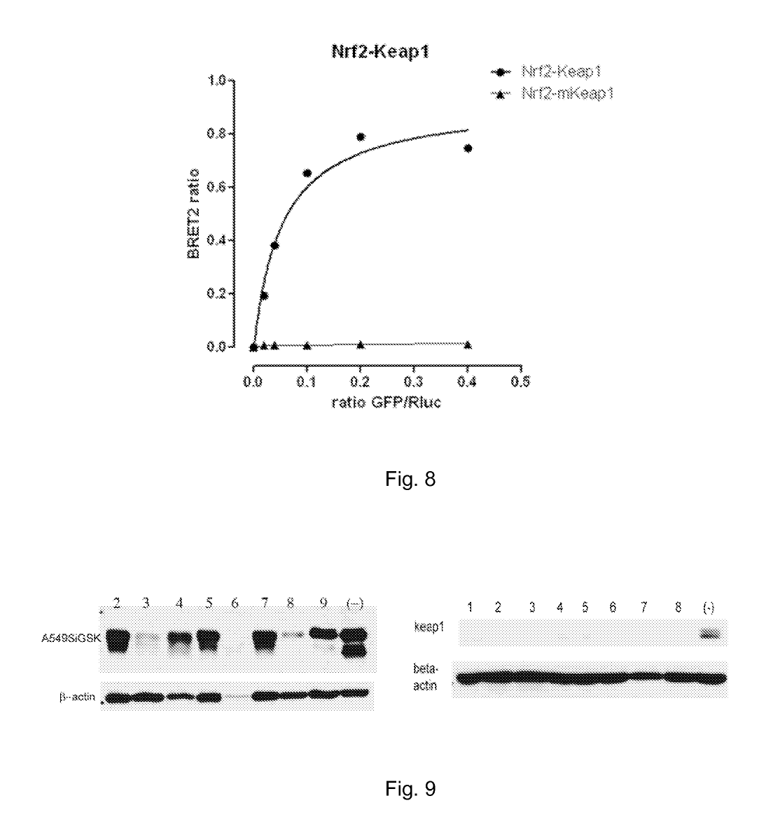

To assess the Nrf2-Keap1 interaction by BRET2 assay, a titration curve is generated by co-transfecting the cells with a fixed amount of Nrf2-Rluc (500 ng), and a range of Keap1-GFP2 amounts (10, 50, 100, 500 and 1000 ng) (FIG. 8). The BRET2 signal increases with the increase in the ratio of GFP2/Rluc, and reaches a plateau when all the energy donor proteins (Rluc-Nrf2) are saturated with the energy acceptor (GFP2-Keap1). With a specific protein-protein interaction, the BRET ratio increases hyperbolically and rapidly saturates (upper curve; specific BRET), while in the case of nonspecific interaction resulting from random collisions, the “bystander BRET” signal increases almost linearly and may saturate at very high expression levels of the energy acceptor (lower curve FIG. 8)

A BRET2₅₀ is calculated from a BRET2 saturation curve, giving a relative affinity index between the test proteins. Keap1-Nrf2 interaction is assessed under different conditions, i.e in the presence of inducer/inhibitor of this interaction such as NR16 and derivatives, or in the presence of Keap1/Nrf2 mutation that disrupt this interaction. NR16 and its derivatives are believed to enhance mutant Keap binding to Nrf2. A change in the affinity between Keap1 and Nrf2 results in a shifted curve and a change in BRET2₅₀. To be certain that NR16 is not in some way re-associating these proteins in a manner that is not identified by BRET, co-immunoprecipitation (co-IP) experiments are performed. IP is performed with anti-Nrf2 antibodies, and immunoblot the precipitate for Keap1, and the reverse is also performed: IP with anti-Keap antibody and detect Nrf2.

Cytotoxicity assays are performed to determine whether a non-toxic dose of NR16 sensitizes A549 cells to paclitaxel and cisplatin. The dose is optimized by examining cell survival and Nrf2 protein levels. A variety of exposure times, prior to and concurrent with exposure to the cytotoxic agents is tested.

To test NR16 and derivative structures in vivo, studies in groups of 5 mice are performed, where each group is administered different doses of NR16 by intraperitoneal injection, and a maximally tolerated non-toxic dose is determined. Toxicity is determined by weight and elevation of liver function tests, which is tested weekly. H460 xenografts are established in mice, and groups of 5 mice are treated at this dose, to then sacrifice them and harvest tumor at 1h, 4h, 8h, 12h after dosing. The tumor are dissected free of fat and subcutaneous tissue, and both fixed for immunohistochemistry and homogenized for Western blotting to measure Nrf2 levels, as well as the level of Nrf2-regulated proteins. These are compared to tumors from non-treated mice. This experiment also provides an opportunity to examine ‘selectivity’ of the sensitization effect, since there is a great degree of homology between mouse and human Keap1 and Nrf2. Therefore liver and bone marrow are also harvested from the chemotherapy treated mice to look for any evidence of enhanced toxicity in the presence of PF-04691502.

### Validation of the Keap1/PI3K Mutation/Amplification Signature as a Biomarker

To further test the specific cytotoxicity of PF-04691502, H460 cells are transfected with an siRNA specifically designed to deplete GSK-3R levels. Another siRNA that depletes Keap1 is used to test whether cells which have an activating mutation of PI3K, but WT Keap1, (e.g. MCF7), are sensitized by PF-04691502 when Keap1 is depleted. The pSuper platform (OligoEngine, Seattle, Wash.) is used to clone both siRNAs. Small inhibitory RNA sequences are designed using software available online at invitrogen.com. The siRNA, when dimerized form Bgl II and Hind III restriction sites. The oligos are cloned into the Bgl II/Hind III sites of the pSuper-puro vector. Clones transfected with either siRNAs are shown in FIG. 9.

These experiments confirm that the proposed mechanism of sensitization is GSK-dependent in Keap1 mutated or depleted (siRNA) cells. Cells are exposed to PF-04691502 50nM for 24 h, at which time media is replaced with fresh PF-04691502 and one of either cisplatin or paclitaxel is added at various doses. Cell survival is determined using Alamar blue. The shift in IC₅₀ is determined. In H460 cells, depletion of GSK-3β has no effect; in MCF7 cells, Keap1 depletion causes a sensitizing effect of PF-04691502, since these cells bear PI3K mutations only.

In addition to the effects of PF-04691502 on Nrf2 protein levels, Nrf2 function is determined by measuring the expression of some of the Nrf2-regulated genes. RT-PCR is used to measure the mRNA levels of a number of Nrf2-regulated genes, including ABCC1, GCS and others. For each gene, cDNA is generated from RNA using primers and Superscript II reverse transcriptase (Invitrogen). Gene expression is determined using Power SYBR green master mix (Applied Biosystems) with primers designed using the Primer Express 3.0. GAPDH is the endogenous control, measured with a VIC-labeled Taqman probe and Fas Master Mix. QPCR is done on the 7500 Fast Real-time PCR system and analyzed using relative quantities with untransfected cells as the calibrator.

Since phospho-GSK is a target, the same scenario is expected to occur in LnCAP prostate cancer cells, which comprise a Keap1 mutation. These cells lack PI3K mutation, but by virtue of a PTEN mutation, they have constitutively activated Akt. Thereof, if GSK-3β is phosphorylated, the specific Akt inhibitor Perifosine, currently in clinical trials, also depletes phospho-GSK 3β, Nrf2 and results in chemosensitization.

### In Vivo Testing

The H460 NSCLC cells are tested and the findings are validated in other cell lines with somatic changes in both Keap1 and PI3K as identified below. The cells are xenografted by injecting 10⁶ cells subcutaneously in the flanks of each of 10 animals for each dose and time of exposure. In mouse xenograft models, oral dosing of PF-04691502 once daily at 10 mg/Kg results in significant depletion of phosphorylated forms of proteins in the PI3K pathway such as pAkt (and pGSK-3β), which persists for up to 8 hours or more. In H460 xenograft mice the level of phospho-GSK-3R is determined, and Nrf2 and Nrf-regulated proteins in tumor samples are measured using both IHC and Western blotting of tumor lysates. In a separate group of such mice, the dose of cisplatin (IP) that results in significant tumor growth delay or shrinkage, without causing significant toxicity (e.g. loss of body weight) is determined and compared to a solvent control.

To confirm the role of GSK-3β in in vivo experiments, siRNAs that stably and significantly reduce GSK-3β are generated. Cells (10⁶) are stably transfected with this siRNA vs. an empty vector are injected into either flank of the same mouse. The mice are then exposed to PF-04691502 using the optimized dose and exposure time determine in above, and both tumors are measured 3 times/week. If GSK-3β mediates the sensitizing effect of PF-04691502, treatment of animals with the ‘effective’ dose of cisplatin determined above results in greater efficacy, seen as greater tumor growth delay or a decrease in tumor volume in the tumors transfected with the empty vector, compared to the siRNA transfected cells.

### Large Scale Screening of Biobanks for Keap1/PI3K Molecular Signature

Alteration in PI3Kinase signaling pathway is frequent in a variety of human tumors. Although the strategy used can be relevant to any scenario in which GSK-3β phosphorylation would be exaggerated, PI3K activating mutations and increased copy number are initially examined, which together are seen in at least 10-30% of many tumor types. What is less known is the frequency of Keap1 somatic mutations in general. The frequency of somatic genetic changes in both genes in the same cancer cell line or tumor type is unknown. To answer this question 50 human tumor cell lines of various tissues of origin that have been shown to have activated PI3K pathway signaling are screened for Keap1 mutation to find an additional 3 cell lines of various tumor types to validate this ‘gene signature’, which in the NSCLC cell line H460 predicts a benefit of adding PF-0469150 to cytotoxic chemotherapy. For example, LnCAP prostate cells bear both mutations resulting in Akt activation and a known Keap1 mutation. Although these various cell lines have different genetic backgrounds, it is believed that they should be relatively more resistant to chemo drugs alone compared to cells without these mutations, and to be sensitized to the cytotoxic chemotherapy by the addition of either NR16 or derivatives, to the PI3K pathway inhibitors (PI3K or Akt-specific).

Annotated biobanks are screened for Keap1 mutations across a number of tumor types, beginning with NSCLC, for which >500 samples are available, including 68 of the papillary carcinoma sub-type. Banks of ovarian and prostate cancer are also screened, with a threshold of at least 5%. Banks of 200 samples achieve an adequate confidence level and include the most common sub-categories of each tumor (i.e. grade, stage, etc.). Starting with lung cancer, in each tumor sample found to have a Keap1 mutation, the PI3Kinase signaling pathway is subsequently examined, beginning with PI3K mutation (by High Resolution Melting followed by sequence analysis) or increased copy number (using aGCH, performed in the Segal biomarker core). In subsequent studies, and as the mechanistic work evolves, other genes in the PI3K pathway that contribute to its activation (e.g. Akt mutations, etc.) are to be examined.

Ultimately these genes are examined in tumor samples from patients receiving combination PI3K inhibitor and chemotherapy. Recent in vitro studies suggest synergy in some cell lines, but combination clinical trials are just starting and do not include biopsy of the metastatic tumor being treated (available tissue is from the primary tumor, often resected in the past and followed by a variety of other treatments prior to this trial). For now, the relation between Keap1 mutations may be examined and clinical response to a platinum agent in a tumor besides NSCLC. The molecular signature of therapeutic resistance to a treatment with an oxaliplatin-based front-line treatment of metastatic colorectal cancer comprises frequent activating mutations in Akt. Biopsies of the metastatic tumor are taken prior to treatment with this oxaliplatin-based front-line treatment of metastatic colorectal cancer and at the time of clinical resistance (tumor growth). The specimens are collected in a manner that preserves nucleic acids and proteins for analysis. The A549 cells are as resistant to oxaliplatin and carboplatin, and this represents a unique opportunity to examine the relation between Keap1 mutation and therapeutic response in the same clinical specimen.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A mutation-selective chemosensitizer comprising a compound of formula (I) for opening a mutated Nrf2 binding site of a mutated keap1 protein to restore interaction between said mutated keap1 protein and a Nrf2 protein: 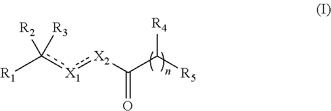
wherein
R₁ is a heterocyclic aromatic or non-aromatic 5 to 8-membered ring or double-ring containing 1-8 heteroatoms selected from N, O or S,
wherein N and S can be oxidized and N can be quaternized, and any atom of said ring can be substituted with a group chosen from
i. halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
ii. heterocyclic aromatic or non-aromatic 5- to 10-membered ring containing 1-4 heteroatoms selected from N, O or S, wherein N and S can be oxidized and N can be quaternized,
iii. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂, 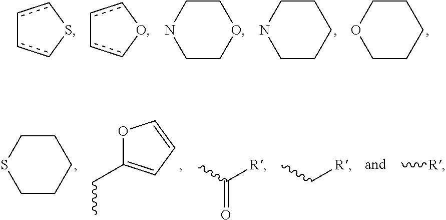
wherein R′ is chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
R₅ is selected from
iv. hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
v. heterocyclic aromatic or non-aromatic 5- to 10-membered ring containing 1-4 heteroatoms selected from N, O or S, wherein N and S can be oxidized and N can be quaternized,
vi. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂, 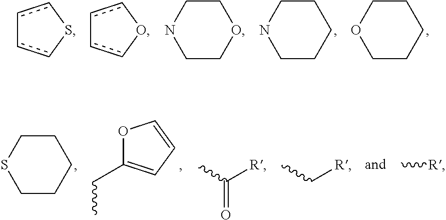
wherein R′ is chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
vii. 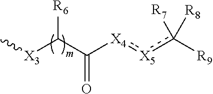
R₂, R₃, R₄, R₆, R₇, and R₈ are independently chosen or identical, and are chosen from
viii. hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
ix. heterocyclic aromatic or non-aromatic 5- to 10-membered ring containing 1-4 heteroatoms selected from N, O or S, wherein N and S can be oxidized and N can be quaternized,
x. ═O, CH₃, OCH₃, OC₂H₅, NO₂, CN, F, Cl, Br, SH, CF₃, OCF₃, O(CF₂)₂H, NH₂, N(CH₃)₂, N(C₂H₄OH)₂, CH(OC₂H₅)₂, 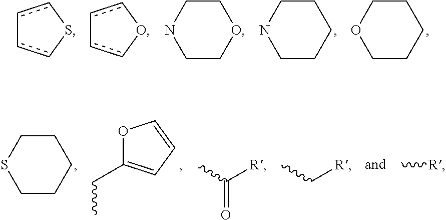
wherein R′ is chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
R₉ is a heterocyclic aromatic or non-aromatic 5 to 8-membered ring or double-ring containing 1-8 heteroatoms selected from N, O or S,
wherein N and S can be oxidized and N can be quaternized, and any atom of said ring can be substituted with a group chosen from
xi. halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
xii. heterocyclic aromatic or non-aromatic 5- to 10-membered ring containing 1-4 heteroatoms selected from N, O or S, wherein N and S can be oxidized and N can be quaternized,
xiii. ═O, CH₃, OCH₃, OC₂H₅, NO₂, ON, F, Cl, Br, SH, CF₃, OCF₃, O(CF₂)₂H, NH₂, N(CH₃)₂, N(C₂H₄OH)₂, CH(OC₂H₅)₂, 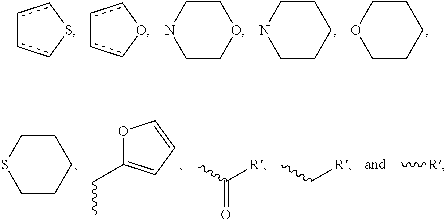
wherein R′ is chosen from hydrogen, halide, hydroxyl, alkyl, substituted alkyl, alkenyl, substituted alkenyl, aryl, substituted aryl, alkyloxide, substituted alkyloxide, halogenated alkyl, halogenated alkenyl, halogenated alkyloxide, halogenated substituted alkyloxide, amine, substituted amine, cycloalkyl, substituted cycloalkyl,
X₁, X₂, X₄ and X₅ are independently chosen or identical and are selected from N, NH, C, CH, or CH₂,
X₃ is selected from N, O, or S,
n and m are independently chosen or identical and can be 1 to 10 C atom,
wherein [symbol] indicates an attachment point, and [symbol]
is a single bond or a double bond;
pharmaceutically acceptable salt, racemic mixture, enantiomer, diastereoisomer, isomer, and tautomer thereof.

2. The mutation-selective chemosensitizer of claim 1, wherein said compound is a compound of formula (II): 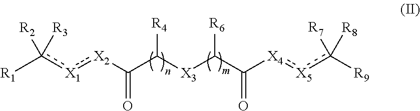
wherein
R₁, R₂, R₃, R₄, R₅, R₆, R₇, R₈, R₉, X₁, X₂, X₃, X₄, and X₅ are as defined in claim 1.

3. The mutation-selective chemosensitizer of claim 1, wherein said compound corrects a Keap1 mutation to restore interaction between a mutated Keap1 protein and said Nrf2 protein.

4. The mutation-selective chemosensitizer of claim 2, wherein said compound of formula (II) is

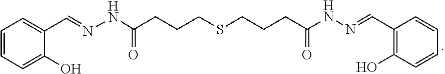 [SMILES: O=C(CCCSCCCC(=O)N/N=C/c1ccccc1O)N/N=C/c1ccccc1O]

5. The mutation-selective chemosensitizer of claim 2, wherein said compound of formula (II) is

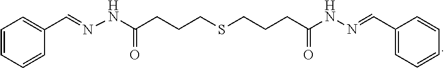 [SMILES: O=C(CCCSCCCC(=O)N/N=C/c1ccccc1)N/N=C/c1ccccc1]

6. The mutation-selective chemosensitizer of claim 2, wherein said compound of formula (II) is

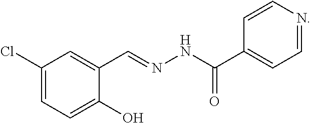 [SMILES: O=C(N/N=C/c1cc(Cl)ccc1O)c1ccncc1]

7. The mutation-selective chemosensitizer of claim 2, wherein said compound of formula (II) is

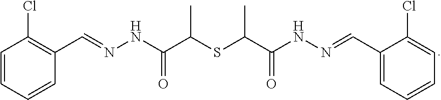 [SMILES: CC(SC(C)C(=O)N/N=C/c1ccccc1Cl)C(=O)N/N=C/c1ccccc1Cl]

8. The mutation-selective chemosensitizer of claim 2, wherein said compound of formula (II) is

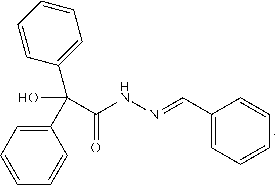 [SMILES: O=C(N/N=C/c1ccccc1)C(O)(c1ccccc1)c1ccccc1]

9. The mutation-selective chemosensitizer of claim 2, wherein said compound of formula (II) is

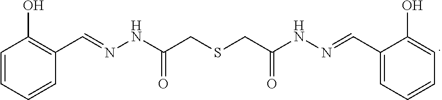 [SMILES: O=C(CSCC(=O)N/N=C/c1ccccc1O)N/N=C/c1ccccc1O]

10. A pharmaceutical composition for the inhibition of a Nrf2 protein which comprises a therapeutically effective amount of a compound of formula (I) as defined in claim 1, in association with a pharmaceutically acceptable carrier.

11. A pharmaceutical composition for overcoming drug resistance in cancer chemotherapy and for the inhibition of tumor growth which comprises a therapeutically effective amount of a compound of formula (I) as defined in claim 1, in association with a pharmaceutically acceptable carrier.

12. A method of treating and/or preventing a disease which involves the abnormal activation or expression of a Nrf2 protein comprising administering a therapeutically effective amount of the compound of formula (I) as defined in of claim 1.

13. A method of treating a cancer in a subject in need thereof comprising administering a therapeutically effective amount of a compound of formula (I) as defined in of claim 1.

14. The method of claims 13, wherein said cancer is chosen from liver cancer, lung cancer, breast cancer, prostate cancer, colon cancer, neuroblastoma or leukemia.
